(19) United States
(12) Patent Application Publication
(10) Pub. No.: US 2022/0041532 A1
(43) Pub. Date: Feb. 10, 2022

(54) HYDROFLUOROOLEFINS AND METHODS OF USING SAME

(71) Applicant: 3M INNOVATIVE PROPERTIES COMPANY, St. Paul, MN (US)

(72) Inventors: Sean M. Smith, Woodbury, MN (US); Michael G. Costello, Afton, MN (US); Michael J. Bulinski, Stillwater, MN (US); Hui Ren, Woodbury, MN (US); Forrest A. Coughlin, Chaska, MN (US)

(21) Appl. No.: 17/414,686

(22) PCT Filed: Dec. 19, 2019
(86) PCT No.: PCT/IB2019/061118

§ 371 Date: Jun. 16, 2021

Related U.S. Application Data
(60) Provisional application No. 62/782,476, filed on Dec. 20, 2018.

(51) Int. Cl. C07C 21/18; C11D 11/00; C11D 7/30

(52) U.S. Cl. CPC C07C 21/18; C11D 7/30; C11D 11/0047

(57) ABSTRACT

A hydrofluoroolefin compound represented by the following general formula (II): Formula (II) where Rf is a linear, branched, or cyclic perfluoroalkyl group having 1-6 carbon atoms, and optionally comprises at least one catenated heteroatom selected from nitrogen or oxygen; n is 0 or 1; X is Cl or Br; with the following proviso: when Rf is CF3, then n is 1.

### FIELD

The present disclosure relates brominated or chlorinated hydrofluoroolefins and methods of making and using the same, and to working fluids that include the same.

### BACKGROUND

Various hydrofluoroolefin compounds are described in, for example, Md. J. Alam et al., International Journal of Refrigeration 2018, 90, 174-180, U.S. Pat. App. Pub. 2017/0369668, and U.S. Pat. No. 8,642,819.

### SUMMARY

In some embodiments, a composition is provided. The composition includes a hydrofluoroolefin represented by the following structural formula (I):

(H)ₙ—R_{f}—(CFH)ₘ—CF═CHX (I)

R_{f} is a linear, branched, or cyclic perfluoroalkyl group having 1-6 carbon atoms, and optionally comprises at least one catenated heteroatom selected from nitrogen or oxygen;
n is 0 or 1; m is 0 or 1; m+n=0 or 1; and X is Cl or Br;
with the following provisos: when X is Cl and Rf is CF3, then m is 1; when X is Br and Rf is CF₃, then m is 1; and when Rf is cyclic, then m+n=0. The composition further includes a contaminant. The hydrofluoolefin is present in the composition at an amount of at least 25% by weight, based on the total weight of the composition.

In some embodiments, a hydrofluoroolefin compound is provided. The composition includes a hydrofluoroolefin represented by the following general formula (II):

R_{f}(CFH)ₙCF═CHX (II)

R_{f} is a linear, branched, or cyclic perfluoroalkyl group having 1-6 carbon atoms, and optionally comprises at least one catenated heteroatom selected from nitrogen or oxygen; n is 0 or 1; X is Cl or Br; with the following proviso: when Rf is CF3, then n is 1.

In some embodiments, a process for removing a contaminant from a substrate is provided. The process includes contacting the substrate with a hydrofluoroolefin represented by the following structural formula (I):

(H)ₙ—R_{f}—(CFH)ₘ—CF═CHX (I)

R_{f} is a linear, branched, or cyclic perfluoroalkyl group having 1-6 carbon atoms, and optionally comprises at least one catenated heteroatom selected from nitrogen or oxygen; n is 0 or 1; m is 0 or 1; m+n=0 or 1; and X is Cl or Br; with the following provisos: when X is Cl and Rf is CF3, then m is 1; when X is Br and Rf is CF3, then m is 1; and when Rf is cyclic, then m+n=0. The contaminant includes a long chain hydrocarbon alkane.

The above summary of the present disclosure is not intended to describe each embodiment of the present disclosure. The details of one or more embodiments of the disclosure are also set forth in the description below. Other features, objects, and advantages of the disclosure will be apparent from the description and from the claims.

### DETAILED DESCRIPTION

The ever-increasing demand for reliability, continuing miniaturization, and the growing number of faults in electronic components manufactured in no-clean processes all combine to put increased focus on the use of cleaning solvents in electronics manufacturing. There has been rapid growth in the electronics industry on account of the swiftly rising demand for industrial as well as consumer electronics products. Cleaning solvents are specially engineered to dependably dissolve common manufacturing greases and oils (e.g., hydrocarbons having the formula CₙH₂ₙ+2) used in the production of such industrial and consumer electronics products. Fluorinated cleaning solvents which demonstrate high levels of hydrocarbon solubility are suitable for such applications, in part, due to their low flammability, high density, low viscosity, low surface tension, and higher vapor pressure resulting in quick evaporation from components after use. Furthermore, in sharp contrast to hydrocarbon solvents, fluorinated cleaning solvents minimize the amount of residue left on components after cleaning.

Currently, fluids used for dissolving and removing such greases and oils (i.e., long chain hydrocarbons), or other organics from surfaces contain fluid blends that include, for example, trans-di-chloro-ethylene, 1,1,1-trichloroethane (TCA), trichloroethylene, and dichloromethane. Regarding such fluid blends, one drawback to this approach is the tendency for a change in the composition ratio over the lifetime of the cleaning fluid. This change in composition ratio, in turn, leads to safety concerns and also compromises the performance of the cleaning fluid. Therefore, a single composition cleaning fluid which is nontoxic, nonflammable, and high in hydrocarbon solubility would be of significant benefit to the electronics cleaning industry. Moreover, some of the materials currently employed are regulated by the Montreal Protocol as ozone depleting substances or have toxicity concerns.

In view of an increasing demand for environmentally friendly and low toxicity chemical compounds, in addition to strong cleaning ability, there exists a need for new long chain hydrocarbon alkanes cleaning fluids that provide low environmental impact and toxicity. Moreover, such cleaning fluids, ideally, should be functional as a single molecule (as opposed to a blend) and possess a broad boiling point range. Finally, such cleaning fluids should be capable of being manufactured using cost-effective methods.

Generally, the present disclosure provides a new class of compounds useful as cleaning fluids (or as components of cleaning fluids). The compounds are brominated or chlorinated hydrofluoroolefins (HFOs), which provide similar or better cleaning and physical properties to existing cleaning fluids, but generally provide lower atmospheric lifetimes and global warming potentials to provide a more acceptable environmental profile. Furthermore, the brominated or chlorinated hydrofluoroolefins of the present disclosure can function as a single molecule (as opposed to a blend), possess a broad boiling point range (e.g., 30 to 150 degrees Celsius), and can be manufactured cost-effectively.

As used herein, “catenated heteroatom” means an atom other than carbon (for example, oxygen, nitrogen, or sulfur) that is bonded to at least two carbon atoms in a carbon chain (linear or branched or within a ring) so as to form a carbon-heteroatom-carbon linkage.

As used herein, “halogenated” (for example, in reference to a compound or molecule, such as in the case of “halogenated HFO”) means that there is at least one carbon-bonded halogen atom.

As used herein, “fluoro-” (for example, in reference to a group or moiety, such as in the case of “fluoroalkylene” or “fluoroalkyl” or “fluorocarbon”) or “fluorinated” means (i) partially fluorinated such that there is at least one carbon-bonded hydrogen atom, or (ii) perfluorinated.

As used herein, “perfluoro-” (for example, in reference to a group or moiety, such as in the case of “perfluoroalkylene” or “perfluoroalkyl” or “perfluorocarbon”) or “perfluorinated” means completely fluorinated such that, except as may be otherwise indicated, there are no carbon-bonded hydrogen atoms replaceable with fluorine.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure is directed to a hydrofluoroolefin represented by the following structural formula (I):

(H)ₙ—R_{f}—(CFH)ₘ—CF═CHX (I),

where R_{f} is a linear, branched, or cyclic perfluoroalkyl group having 1-6, 1-5, 1-4, 1-3, or 1-2 carbon atoms, and optionally comprises at least one catenated heteroatom selected from nitrogen or oxygen;

n is 0 or 1;

m is 0 or 1;

m+n=0 or 1; and

X is Cl or Br;

with the following provisos:

when X is Cl and Rf is CF₃, then m is 1;

when X is Br and Rf is CF₃, then m is 1; and

when Rf is cyclic, then m+n=0.

In some embodiments, particular hydrofluoroolefins within structural formula (I) may include hydrofluoroolefins having the following structural formulas:

CF2HCF2CF2CF═CHC1; (IA)

or

CF2H(CF2)ₙCF═CHBr (IB)

where n is 0 or 2.

In some embodiments, the present disclosure is directed to a hydrofluoroolefin represented by the following structural formula (II):

R_{f}(CFH)ₙCF═CHX (II)

where R_{f} is a linear, branched, or cyclic perfluoroalkyl group having 1-6, 1-5, 1-4, 1-3, or 1-2 carbon atoms, and optionally comprises at least one catenated heteroatom selected from nitrogen or oxygen; and

n is 0 or 1;

X is Cl or Br;

with the following proviso:

when R_{f} is CF3, then n is 1.

In some embodiments, particular hydrofluoroolefins within structural formula (II) may include hydrofluoroolefins having the following structural formulas:

RfCF═CHCl (IIA)

where R_{f} is a linear, branched, or cyclic perfluoroalkyl group having 2-6, 2-5, 2-4, or 2-3 carbon atoms, and optionally comprises at least one catenated heteroatom selected from nitrogen or oxygen;

RfCF═CHCl (IIB)

where R_{f} is a perfluoroalkyl group having 2-3 carbon atoms;

RfCF═CHBr (IIC)

where R_{f} is a linear, branched, or cyclic perfluoroalkyl group having 2-6, 2-5, 2-4, or 2-3 carbon atoms, and optionally comprises at least one catenated heteroatom selected from nitrogen or oxygen; or

RfCF═CHBr (IID)

where R_{f} is a perfluoroalkyl group having 2-3 carbon atoms.

For purposes of the present disclosure, it is to be appreciated that any of the hydrofluoroolefin compounds may include the E isomer, the Z isomer, or a mixture of the E and Z isomers, irrespective of what is depicted in any of the general formulas or chemical structures.

In some embodiments, any of the above discussed catentated heteroatoms may be secondary O heteroatoms wherein the O is bonded to two carbon atoms. In some embodiments, any of the above discussed catenated heteroatoms may be tertiary N heteroatoms wherein the N is bonded to three perfluorinated carbon atoms.

In some embodiments, any of the above the hydrofluoroolefins may possess excellent hydrocarbon solubility, rendering them highly suitable for use as cleaning solvents. In this regard, in some embodiments, any of the above described hydrofluoroolefins may have a solubility factor defined as follows:

Solubility Factor (SF)=((LSH/14)−1)−3.5((T−70)/70)²+0.643

where LSH is determined in accordance with the Largest Soluble Hydrocarbon Test of the Examples of the present disclosure and T is the normal boiling point of the fluid (in degrees Celsius). In some embodiments, the LSH of the hydrofluoroolefins may be from 14 to 25, 17 to 23, or 17 to 21, in whole number increments. In some embodiments, any of the above described hydrofluoroolefins may have a solubility factor (SF) of greater than 0, greater than 0.1, 0.2, 0.5, 1.0, 1.1, or greater than 1.2.

In some embodiments, the fluorine content in the hydrofluoroolefin compounds of the present disclosure may be sufficient to make the compounds non-flammable according to ASTM D-3278-96 e-1 test method (“Flash Point of Liquids by Small Scale Closed Cup Apparatus”).

In various embodiments, representative examples of the compounds of general formula (I) include the following:

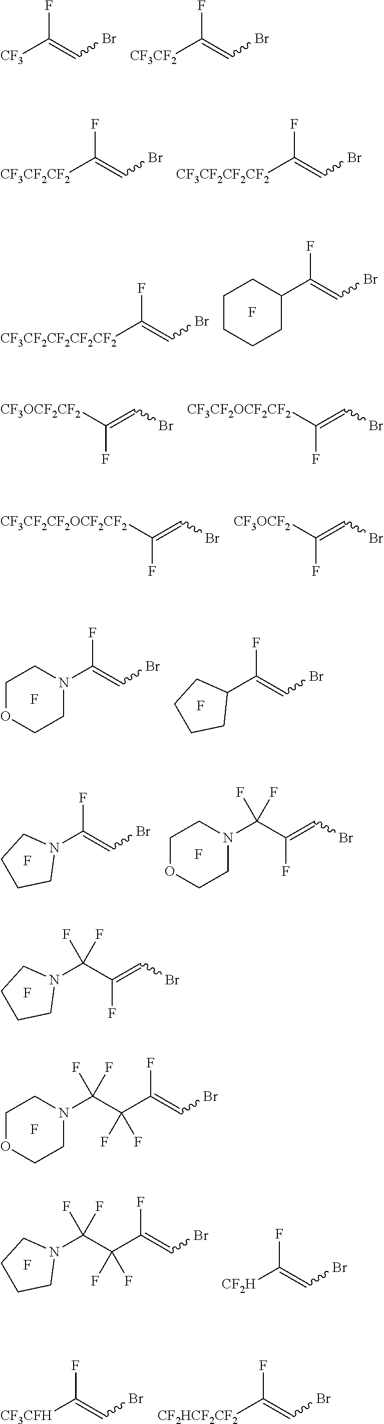

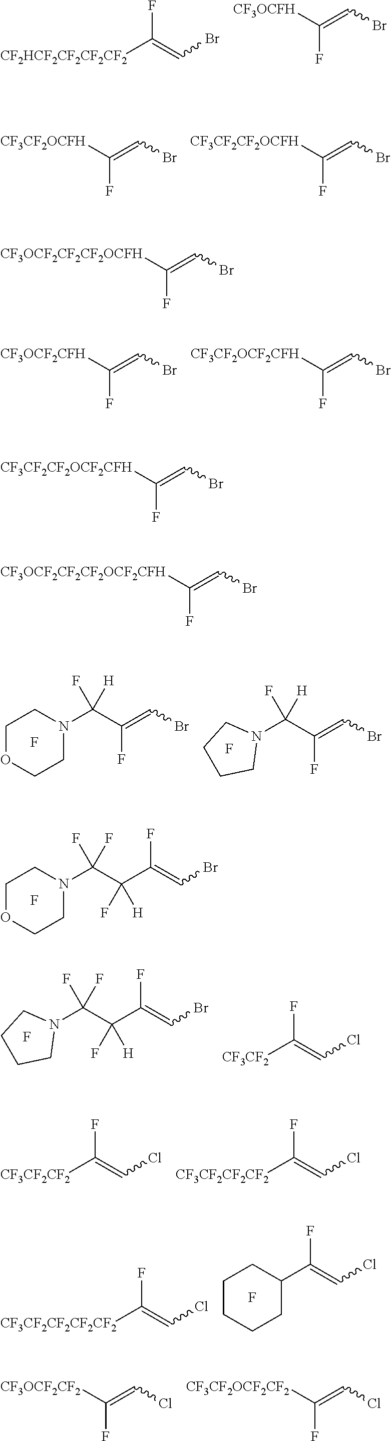

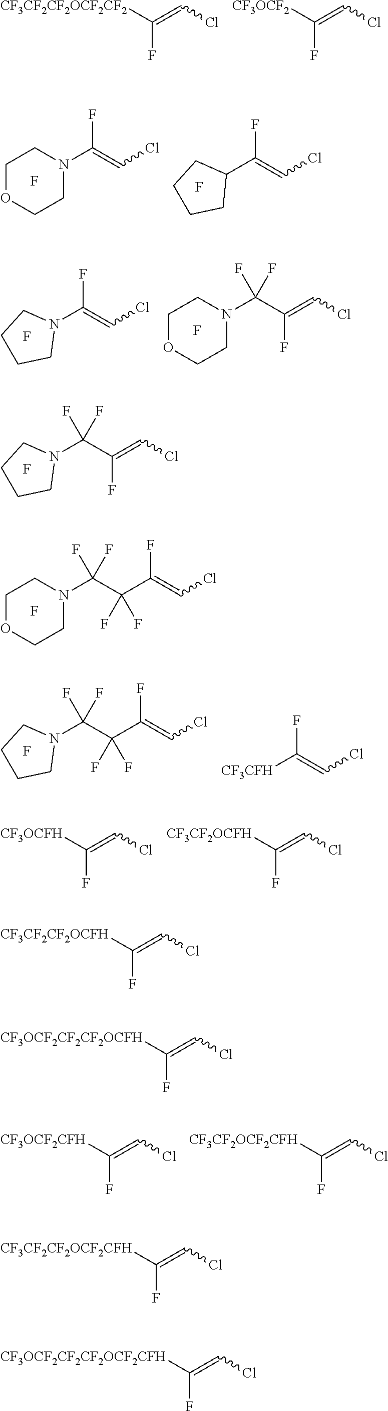

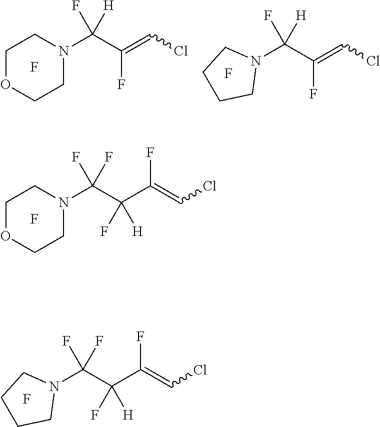

In various embodiments, representative examples of the compounds of general formula (II) include the following:

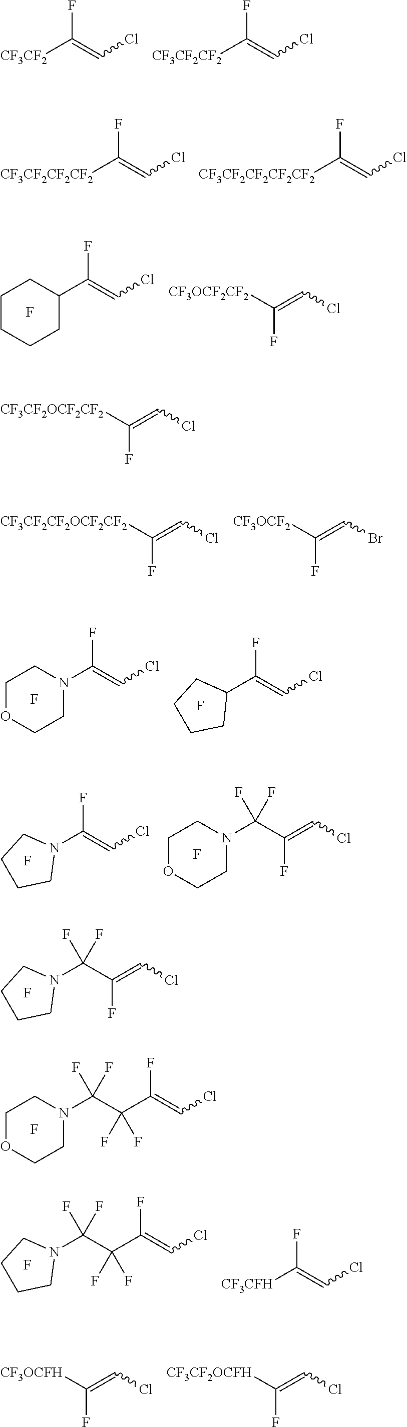

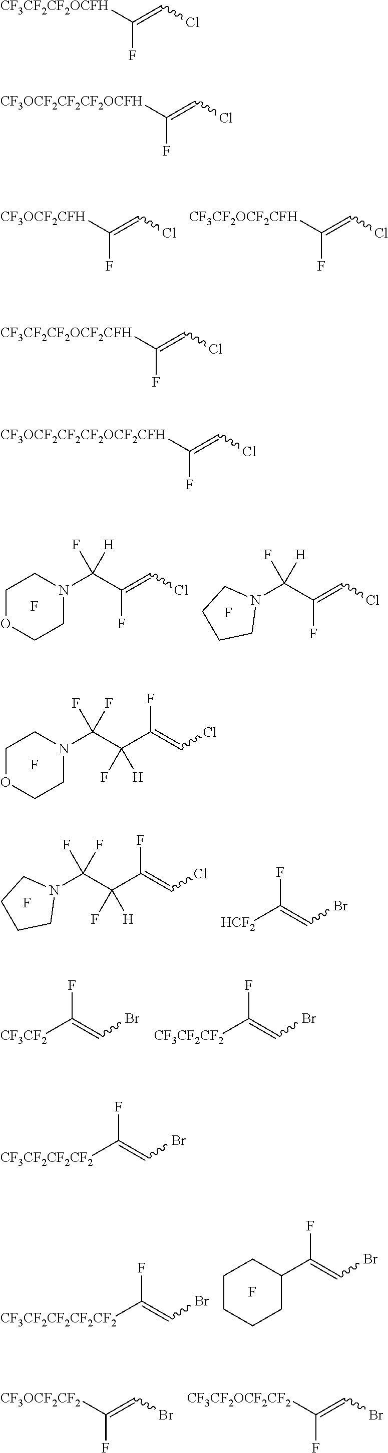

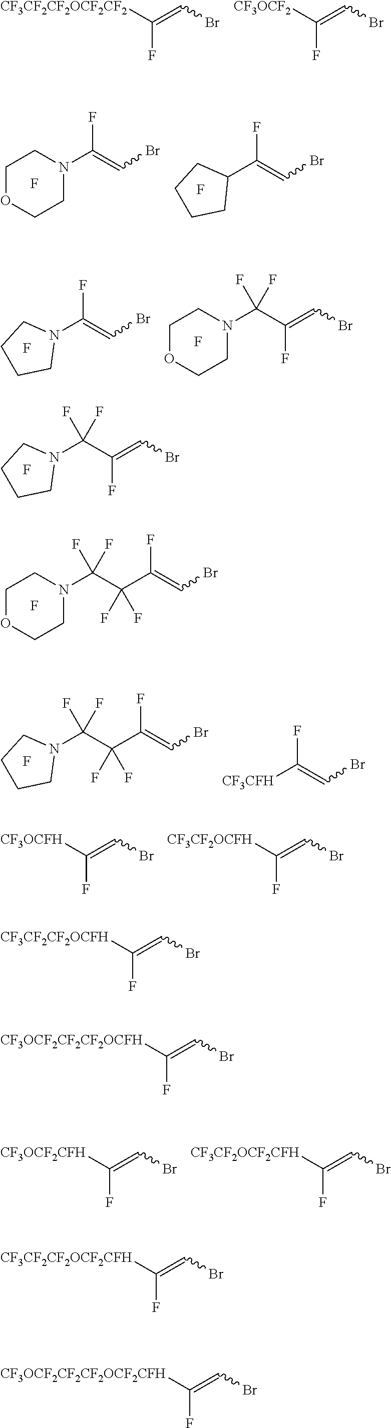

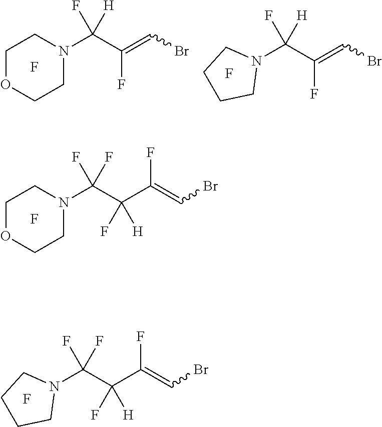

In some embodiments, the hydrofluoroolefins of the present disclosure may be useful over a broad operating temperature range. In this regard, in some embodiments, the hydrofluoroolefins of the present disclosure may have a boiling point of no lower than 30, 40, or 50 degrees Celsius and no higher than 150, 140, 130, 120, 110, 100, 90, or 80 degrees Celsius.

In some embodiments, the hydrofluoroolefins of the present disclosure may be hydrophobic, relatively chemically unreactive, and thermally stable. The hydrofluoroolefin compounds may have a low environmental impact. In this regard, the hydrofluoroolefin compounds of the present disclosure may have a global warming potential (GWP) of less than 200, 150, 100, 50 or less than 10. As used herein, GWP is a relative measure of the global warming potential of a compound based on the structure of the compound. The GWP of a compound, as defined by the Intergovernmental Panel on Climate Change (IPCC) in 1990 and updated in 2007, is calculated as the warming due to the release of 1 kilogram of a compound relative to the warming due to the release of 1 kilogram of CO₂ over a specified integration time horizon (ITH).

$${GWP}_{i} ( {t}^{′} ) = \frac{{\int }_{0}^{ITH} {a}_{i} [ C ( t ) ] d t}{{{\int }^{ITH}}_{0} {a}_{{CO}_{2}} [ {C}_{{CO}_{2}} ( t ) ] dt} = \frac{{\int }_{0}^{ITH} {a}_{i} {C}_{oi} {e}^{- 1 / π} d t}{{\int }_{0}^{ITH} {a}_{{CO}_{2}} [ {C}_{{CO}_{2}} ( t ) ] dt}$$

In this equation aᵢ is the radiative forcing per unit mass increase of a compound in the atmosphere (the change in the flux of radiation through the atmosphere due to the IR absorbance of that compound), C is the atmospheric concentration of a compound, t is the atmospheric lifetime of a compound, t is time, and i is the compound of interest. The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

In some embodiments, the brominated or chlorinated hydrofluoroolefin compounds of the present disclosure can be synthesized by first reducing a perfluorinated acid fluoride with a suitable reducing agent such as NaBH₄ or LiAlH₄ to afford an alcohol. An alcohol can also be prepared by the addition of methanol across a perfluorinated olefin in the presence of a radical initiator (Examples of such initiators include tert-amylperoxy-2-ethylhexanoate (TAPEH, available as LUPEROX 575 from Arkema, Crosby, Tex.), lauryl peroxide, tert-butyl peroxide, tert-amylperoxy-2-ethylhexyl carbonate, and mixtures thereof. The subsequent conversion to a triflate or nonaflate occurs via reaction with CF3SO2F or CF3CF2CF2CF2SO2F in the presence of base (e.g., NaOH, KOH, Na₂CO₃, or K₂CO₃). The resultant triflate or nonaflate can then be converted to the respective chloride or bromide via substitution by LiCl or LiBr, respectively, in a polar aprotic solvent (e.g., DMF, NMP, diethyl ether, THF, Dioxane, diglyme, or tetraglyme). The afforded chloride or bromide is then subjected to aqueous base (e.g., 50% KOH or NaOH) with a catalytic amount of phase-transfer catalyst, such as tetrabutylammonium chloride, to promote dehydrofluorination and give the desired hydrochloro(bromo)fluoroolefin.

In some embodiments, the present disclosure is further directed to working fluids that include the above-described hydrofluoroolefin compounds as a major component. For example, the working fluids may include at least 25%, at least 50%, at least 70%, at least 80%, at least 90%, at least 95%, or at least 99% by weight of the above-described hydrofluoroolefin compounds, based on the total weight of the working fluid. In addition to the hydrofluoroolefin compounds, the working fluids may include a total of up to 75%, up to 50%, up to 30%, up to 20%, up to 10%, or up to 5% by weight of one or more of the following components: alcohols, ethers, alkanes, alkenes, haloalkenes, perfluorocarbons, perfluorinated tertiary amines, perfluoroethers, cycloalkanes, esters, ketones, oxiranes, aromatics, siloxanes, hydrochlorocarbons, hydrochlorofluorocarbons, hydrofluorocarbons, hydrochloroolefins, hydrochlorofluoroolefins, hydrofluoroethers, or mixtures thereof, based on the total weight of the working fluid. Such additional components can be chosen to modify or enhance the properties of a composition for a particular use.

In some embodiments, the present disclosure relates to cleaning compositions that include one or more hydrofluoroolefin compounds of the present disclosure. In use, the cleaning compositions may serve to remove (e.g., dissolve) contaminants from the surface of a substrate. For example, materials such as light hydrocarbon contaminants; higher molecular weight hydrocarbon contaminants such as mineral oils and greases; fluorocarbon contaminants such as perfluoropolyethers, bromotrifluoroethylene oligomers (gyroscope fluids), and chlorotrifluoroethylene oligomers (hydraulic fluids, lubricants); silicone oils and greases; solder fluxes; particulates; water; and other contaminants encountered in precision, electronic, metal, and medical device cleaning can be removed. In some embodiments, the hydrofluoroolefin compounds of the present disclosure may be particularly suited to remove long chain hydrocarbon alkane contaminants.

In some embodiments, the cleaning compositions of the present disclosure may include one or more co-solvents. In some embodiments, the hydrofluoroolefin compounds may be present in the cleaning compositions in an amount of greater than 50 weight percent, greater than 60 weight percent, greater than 70 weight percent, greater than 80 weight percent, greater than 90 weight percent, or greater than 95 weight percent, based upon the total weight of the hydrofluoroolefin compounds and the co-solvent(s).

In illustrative embodiments, the co-solvent may include alcohols, ethers, alkanes, alkenes, haloalkenes, perfluorocarbons, perfluorinated tertiary amines, perfluoroethers, cycloalkanes, esters, ketones, oxiranes, aromatics, haloaromatics, siloxanes, hydrochlorocarbons, hydrochlorofluorocarbons, hydrofluorocarbons, hydrofluoroolefins, hydrochloroolefins, hydrochlorofluoroolefins, hydrofluoroethers, or mixtures thereof. Representative examples of co-solvents which can be used in the cleaning compositions may include methanol, ethanol, isopropanol, t-butyl alcohol, methyl t-butyl ether, methyl t-amyl ether, 1,2-dimethoxyethane, cyclohexane, 2,2,4-trimethylpentane, n-decane, terpenes (e.g., a-pinene, camphene, and limonene), trans-1,2-dichloroethylene, cis-1,2-dichloroethylene, methylcyclopentane, decalin, methyl decanoate, t-butyl acetate, ethyl acetate, diethyl phthalate, 2-butanone, methyl isobutyl ketone, naphthalene, toluene, p-chlorobenzotrifluoride, trifluorotoluene, bis(trifluoromethyl)benzenes, hexamethyl disiloxane, octamethyl trisiloxane, perfluorohexane, perfluoroheptane, perfluorooctane, perfluorotributylamine, perfluoro-N-methyl morpholine, perfluoro-2-butyl oxacyclopentane, methylene chloride, chlorocyclohexane, 1-chlorobutane, 1,1-dichloro-1-fluoroethane, 1,1,1-trifluoro-2,2-dichloroethane, 1,1,1,2,2-pentafluoro-3,3-dichloropropane, 1,1,2,2,3-pentafluoro-1,3-dichloropropane, 2,3-dihydroperfluoropentane, 1,1,1,2,2,4-hexafluorobutane, 1-trifluoromethyl-1,2,2-trifluorocyclobutane, 3-methyl-1,1,2,2-tetrafluorocyclobutane, 1-hydropentadecafluoroheptane, or mixtures thereof. For example, such co-solvents can be chosen to modify or enhance the solvency properties of a cleaning composition for a particular use and can be utilized in ratios (of co-solvent to hydrofluoroolefin compounds) such that the resulting composition has no flash point.

In various embodiments, the cleaning compositions may include one or more surfactants. Suitable surfactants include those surfactants that are sufficiently soluble in the fluorinated olefin, and which promote contaminant removal by dissolving, dispersing, or displacing the contaminant. One useful class of surfactants are those nonionic surfactants that have a hydrophilic-lipophilic balance (HLB) value of less than about 14. Examples include ethoxylated alcohols, ethoxylatedalkyl phenols, ethoxylated fatty acids, alkylarysulfonates, glycerol esters, ethoxylated fluoroalcohols, and fluorinated sulfonamides. Mixtures of surfactants having complementary properties may be used in which one surfactant is added to the cleaning composition to promote oily contaminant removal and another added to promote water-soluble contaminant removal. The surfactant, if used, can be added in an amount sufficient to promote contaminant removal. Typically, surfactant is added in amounts from 0.1 to 5.0 wt. %, or amounts from about 0.2 to 2.0 wt. %, based on the total weight of the surfactant(s) and the hydrofluoroolefin compounds.

In some embodiments, if desirable for a particular application, the cleaning compositions can further include one or more dissolved or dispersed gaseous, liquid, or solid additives (for example, carbon dioxide gas, stabilizers, antioxidants, or activated carbon).

In some embodiments, the present disclosure is further directed to the above-described cleaning compositions, in their post-clean state. In this regard, the present disclosure is directed to any of the above-described cleaning compositions that include one or more dissolved or dispersed (or otherwise contained therein) contaminants such as, for example, any of the above discussed contaminants. In various embodiments, the dissolved or dispersed contaminant may include one or more long chain hydrocarbon alkanes. The dissolved or dispersed contaminants may be present in the post-clean cleaning composition in an amount of between 0.0001% and 0.1 wt. %, between 0.1 and 10 wt. %, or between 10 and 20 wt. %; or at least 5 wt. %, at least 10 wt. %, or at least 20 wt. %, based on the total weight of the hydrofluoroolefin compounds and the contaminants.

In some embodiments, the cleaning compositions of the present disclosure can be used in either the gaseous or the liquid state (or both), and any of known or future techniques for “contacting” a substrate can be utilized. For example, a liquid cleaning composition can be sprayed or brushed onto the substrate, a gaseous cleaning composition can be blown across the substrate, or the substrate can be immersed in either a gaseous or a liquid composition. Elevated temperatures, ultrasonic energy, and/or agitation can be used to facilitate the cleaning. Various different solvent cleaning techniques are described by B. N. Ellis in Cleaning and Contamination of Electronics Components and Assemblies, Electrochemical Publications Limited, Ayr, Scotland, pages 182-94 (1986), which is herein incorporated by reference in its entirety.

Both organic and inorganic substrates can be cleaned by the processes of the present disclosure. Representative examples of the substrates include metals; ceramics; glass; polycarbonate; polystyrene; acrylonitrile-butadiene-styrene copolymer; natural fibers (and fabrics derived therefrom) such as cotton, silk, fur, suede, leather, linen, and wool; synthetic fibers (and fabrics) such as polyester, rayon, acrylics, nylon, or blends thereof, fabrics comprising a blend of natural and synthetic fibers; and composites of the foregoing materials. In some embodiments, the process may be used in the precision cleaning of electronic components (e.g., circuit boards), optical or magnetic media, or medical devices.

In some embodiments, the present disclosure relates to a process for cleaning a substrate. The cleaning process can be carried out by contacting a contaminated substrate with a cleaning composition as discussed above.

### Listing of Embodiments

1. A composition comprising:

a hydrofluoroolefin represented by the following structural formula (I):

following structural formula (I):

(H)ₙ—R_{f}—(CFH)ₘ—CF═CHX (I)

where R_{f} is a linear, branched, or cyclic perfluoroalkyl group having 1-6 carbon atoms, and optionally comprises at least one catenated heteroatom selected from nitrogen or oxygen;

n is 0 or 1;

m is 0 or 1;

m+n=0 or 1; and

X is Cl or Br;

with the following provisos:

when X is Cl and Rf is CF3, then m is 1;

when X is Br and Rf is CF3, then m is 1; and

when Rf is cyclic, then m+n=0; and

a contaminant;

wherein the hydrofluoolefin is present in the composition at an amount of at least 25% by weight, based on the total weight of the composition.

2. The composition of embodiment 1, wherein the contaminant comprises a long chain hydrocarbon alkane.
3. The composition of any one of embodiments 1-2, wherein the hydrofluoroolefin compound has the following general formula (IA):

CF2HCF2CF2CF═CHC1; (IA).

4. The composition of any one of embodiments 1-2, wherein the hydrofluoroolefin compound has the following general formula (IB):

CF2H(CF2)ₙCF═CHBr (IB)

where n is 0 or 2.

5. The composition of any one of embodiments 1-4, wherein the hydrofluoroolefin compound has a solubility factor greater than 0.

### Examples

Objects and advantages of this disclosure are further illustrated by the following illustrative examples. Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, Mo., US or may be synthesized by conventional methods.

The following abbreviations are used herein: mL=milliliters, L=liters, mol=moles, mmol=millimoles, min=minutes, hr=hours, d=days, g=grams, A=Angstroms, μm=micrometers (10⁻⁶ n), ° C.=degrees Celsius, bp=boiling point, mp=melting point. “RT” or “room temperature” refers to an ambient temperature of approximately 20-25° C., with an average of 23° C.

TABLE 1
Materials

| Material | Description | Source |
|---|---|---|
| N(ⁿBu)₄Cl | Tetrabutylammonium | Sigma-Aldrich |
| | chloride | Corp., Saint |
| | | Louis, MO, US |
| Powdered KOH | Powdered Potassium | Sigma-Aldrich |
| | hydroxide | Corp., Saint |
| | (85%) | Louis, MO, US |
| 50% KOH | Potassium hydroxide | Alfa Aesar, |
| | (50%) | Haverhill, MA, |
| | | US |
| 50% NaOH | Sodium hydroxide | Alfa Aesar, |
| | (50%) | Haverhill, MA, |
| | | US |
| NaBH4 | Sodium Borohydride | Sigma-Aldrich |
| | | Corp., Saint |
| | | Louis, MO, US |
| CF₃CH₂OH | 2,2,2-Trifluoroethanol | SynQuest |
| | | Laboratories, |
| | | Alachua FL, US |
| CF₃CF₂CH₂OH | 1H,1H- | Oakwood |
| | Pentafluoropropanol, | Products, Inc., |
| | available from | Augusta, GA, US |
| CF₃CF₂CF₂CH₂OH | 2,2,3,3,4,4,4- | SynQuest |
| | Heptafluorobutanol | Laboratories, |
| | | Alachua FL, US |
| CF₃CFHCF₂CH₂OH | 2,2,3,4,4,4- | SynQuest |
| | Hexafluorobutan-1-ol | Laboratories, |
| | | Alachua FL, US |
| CF₃OCF═CF₂ | Trifluoromethyl | SynQuest |
| | trifluorovinylether | Laboratories, |
| | | Alachua FL, US |
| CF₃OCF₂CF₂C(O)F | Perfluoromethoxy- | Exfluor |
| | propionoyl | Research Corp., |
| | fluoride | Round Rock, TX |
| CF₃CF₂CF₂CF₂CH₂OH | 2,2,3,3,4,4,5,5,5- | SynQuest |
| | Nonafluoropentanol | Laboratories, |
| | | Alachua FL, US |
| CF₂HCF₂CH₂OH | 2,2,3,3- | Daiken Industries, |
| | Tetrafluoropropanol | Osaka, |
| | | Japan |
| CF₂HCF₂CF₂CF₂CH₂OH | 2,2,3,3,4,4,5,5- | Daiken Industries, |
| | Octafluoropentanol | Osaka, |
| | | Japan |
| PBSF | Perfluorobutane sulfonyl | Sigma-Aldrich |
| | fluoride | Corp., Saint |
| | | Louis, MO, US |
| LiBr | Lithium bromide | Alfa Aesar, |
| | | Haverhill, MA, |
| | | US |
| LiCl | Lithium chloride | Alfa Aesar, |
| | | Haverhill, MA, |
| | | US |
| Diglyme | Diethylene glycol | Sigma-Aldrich |
| | dimethyl ether | Corp., Saint |
| | | Louis, MO, US |
| Tetraglyme | Tetraethylene | Alfa Aesar, |
| | glycol dimethyl | Haverhill, MA, |
| | ether | US |
| C-9 hydrocarbon | C₉H₂₀, mp = −53.5° C. | Sigma-Aldrich |
| | | Corp., Saint |
| | | Louis, MO, US |
| C-10 hydrocarbon | C₁₀H₂₂, mp = −29.7° C. | Sigma-Aldrich |
| | | Corp., Saint |
| | | Louis, MO, US |
| C-11 hydrocarbon | C₁₁H₂₄, mp = −25° C. | Sigma-Aldrich |
| | | Corp., Saint |
| | | Louis, MO, US |
| C-12 hydrocarbon | C₁₂H₂₆, mp = −9.6° C. | Sigma-Aldrich |
| | | Corp., Saint |
| | | Louis, MO, US |
| C-13 hydrocarbon | C₁₃H₂₈, mp = −5.4° C. | Sigma-Aldrich |
| | | Corp., Saint |
| | | Louis, MO, US |
| C-14 hydrocarbon | C₁₄H₃₀, mp = 5.9° C. | Sigma-Aldrich |
| | | Corp., Saint |
| | | Louis, MO, US |
| C-15 hydrocarbon | C₁₅H₃₂, mp = 9.9° C. | Sigma-Aldrich |
| | | Corp., Saint |
| | | Louis, MO, US |
| C-16 hydrocarbon | C₁₆H₃₄, mp = 18.2° C. | Sigma-Aldrich |
| | | Corp., Saint |
| | | Louis, MO, US |
| C-17 hydrocarbon | C₁₇H₃₆, mp = 21° C. | Sigma-Aldrich |
| | | Corp., Saint |
| | | Louis, MO, US |
| C-18 hydrocarbon | C₁₈H₃₈, mp = 28-30° C. | Sigma-Aldrich |
| | | Corp., Saint |
| | | Louis, MO, US |
| C-19 hydrocarbon | C₁₉H₄₀, mp = 32-34° C. | Sigma-Aldrich |
| | | Corp., Saint |
| | | Louis, MO, US |
| C-20 hydrocarbon | C₂₀H₄₂, mp = 36.7° C. | Sigma-Aldrich |
| | | Corp., Saint |
| | | Louis, MO, US |
| C-21 hydrocarbon | C₂₁H₄₄, mp = 40.5° C. | Sigma-Aldrich |
| | | Corp., Saint |
| | | Louis, MO, US |
| C-22 hydrocarbon | C₂₂H₄₆, mp = 42° C. | Sigma-Aldrich |
| | | Corp., Saint |
| | | Louis, MO, US |
| C-23 hydrocarbon | C₂₃H₄₈, mp = 48-50° C. | Sigma-Aldrich |
| | | Corp., Saint |
| | | Louis, MO, US |

### Test Methods

Largest Soluble Hydrocarbon (LSH): The LSH of each hydrofluoroolefin compound was determined by mixing the compound with hydrocarbons of varying molecular weight (CₙH₂ₙ₊₂, where n=9 to 24) in a hydrofluoroolefin:hydrocarbon ratio of about 1:1 to 1:2 by weight at room temperature (25° C.) and at 50° C. The LSH value is reported as the value of n in the formula CₙH₂ₙ₊₂ for the longest hydrocarbon which was compatible with the hydrofluoroolefin without exhibiting haze to the naked eye. A larger value of n is interpreted herein to indicate a greater ability of the hydrofluoroolefin to clean hydrocarbons.

Atmospheric lifetime: The atmospheric lifetimes of hydrobromofluoroolefin Examples 1-3 were determined from their rates of reaction with hydroxyl radicals. The pseudo-first order rate for the reaction of the gaseous hydrobromofluoroolefin with hydroxyl radical was measured in a series of experiments relative to reference compounds such as chloromethane and ethane. The measurements were performed in a 5.7 L, heated FTIR gas cell equipped with a polished semiconductor-grade quartz window. An Oriel Instruments UV Lamp, Model 66921 equipped with a 480W mercury-xenon bulb was used to generate hydroxyl radicals by photolyzing ozone in the presence of water vapor. The concentrations of the hydrobromofluoroolefin and the reference compound were measured as a function of reaction time using an I-Series FTIR from Midac Corporation. The atmospheric lifetime was calculated from the reaction rates for the hydrobromofluoroolefin relative to the reference compounds and the reported lifetime of the reference compounds as shown below:

$${τ}_{x} = {τ}_{r} \cdot \frac{{{k}_{r}}_{ }}{{k}_{x}}$$

where τₓ is the atmospheric lifetime of hydrobromofluoroolefin, τᵣ is the atmospheric lifetime of the reference compound, and kₓ and kᵣ are the rate constants for the reaction of hydroxyl radical with hydrobromofluoroolefin and the reference compound, respectively.

### Sample Preparation

### Example 1: 1-bromo-2,3,3,3-tetrafluoroprop-1-ene

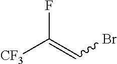

To a 2 L 3-neck round flask equipped with magnetic stirrer, thermocouple, cold water condenser, and an addition funnel were charged pentafluoro-1-propanol (175 g, 1.17 mol), PBSF (360 g, 1.19 mol), and water (400 mL). There was no observed temperature rise during addition of reagents. The addition funnel was then charged with sodium hydroxide (200 g of a 50% solution in water). The 50% sodium hydroxide solution was then added to the stirring mixture dropwise at a rate which kept the internal reaction temperature below 50° C. Once all sodium hydroxide was added, a white hazy mixture was observed. After a 16 hr stir without heating, the resultant reaction mixture was diluted by the addition of water (300 mL). Two layers were observed with white solid at the interface. The bottom layer along with solids were filtered to give a filtrate consisting of mostly fluorochemical layer with some aqueous. The filtrate was then washed with water (300 mL) and the fluorochemical phase was collected to afford 2,2,3,3,3-pentafluoropropyl-1,1,2,2,3,3,4,4,4-nonafluorobutane-1-sulfonate (299 g, 57% yield). GC-FID analysis confirmed 99% purity. The product was stored over 4 Å molecular sieves and was used for the next step without additional purification.

To a 500 mL 3-necked round-bottom flask equipped with a magnetic stir bar, temperature probe, cold water condenser, and addition funnel was charged diglyme (200 mL). Lithium bromide (75.2 g, 866 mmol) was then added in small portions with temperature rises up to 54° C. observed. Once the temperature had dropped to 35° C., 2,2,3,3,3-pentafluoropropyl 1,1,2,2,3,3,4,4,4-nonafluorobutane-1-sulfonate (200.1 g, 440 mmol) was added via the addition funnel at a rate which avoided exceeding 40° C. reaction temperature. After complete addition, the resultant reaction mixture was heated to 60° C. followed by a 2 d stir. The reaction mixture was then cooled to room temperature with stirring followed by the addition of water (200 mL). The fluorous layer was collected and washed again with water (2×100 mL) to give the desired 3-bromo-1,1,1,2,2-pentafluoro-propane (75.5 g, 75 mass %, 60% yield). The isolated material was used without further purification for the next step.

To a two-neck round bottom flask equipped with a water-cooled reflux condenser, magnetic stir bar, and rubber septum were added powdered potassium hydroxide (37.6 g of 85 wt % KOH powder, 570 mmol) and water (70 mL). After addition of water, the temperature had reached 65° C. The solution was allowed to cool to 35° C. with stirring before the addition of tetrabutylammonium chloride (5.0 g, 18.0 mmol). 3-Bromo-1,1,1,2,2-pentafluoropropane (95.1 g at 78 wt % purity, 348 mmol) was then added dropwise to the stirring mixture solution at 35° C. over the course of 15 minutes via syringe. The resultant amber mixture was then allowed to stir for one hour at the same temperature. GC-FID analysis of the fluorochemical phase indicated approximately 92% conversion of starting material. The reaction was allowed to stir overnight at 35° C. The resultant reaction mixture was then allowed to cool to room temperature followed by the addition of 100 mL water. The fluorous phase was separated and analyzed by GC-FID which indicated a mixture containing 98% of the desired 1-bromo-2,3,3,3-tetrafluoroprop-1-ene. Concentric tube distillation (34° C., 740 mm/Hg) afforded the desired 1-bromo-2,3,3,3-tetrafluoroprop-1-ene (42.4 g, 62% yield) as a colorless liquid. The identity of the purified composition was confirmed by GC-MS analysis.

### Example 2: 1-bromo-2,3,3,4,4,4-hexafluorobut-1-ene

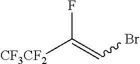

To a 2 L 3-neck round flask equipped with a magnetic stir bar, temperature probe, cold water condenser, and an addition funnel were charged heptafluoro-1-butanol (198.7 g, 993.4 mmol), PBSF (300.1 g, 993.4 mmol), and water (400 mL). There was no observed temperature rise during addition of reagents. The addition funnel was then charged with potassium hydroxide (167.2 g of a 50% solution in water). The potassium hydroxide solution was then added to the stirring mixture dropwise at a rate which kept the internal reaction temperature below 43° C. Once all potassium hydroxide was added, a white hazy mixture was observed. After a 16 hr stir without heating, the resultant reaction mixture was diluted by the addition of water (300 mL). Two layers were observed with white solid at the interface. The bottom layer along with solids were filtered to give a filtrate consisting of mostly fluorochemical layer with some aqueous. The filtrate was then washed with water (300 mL) and the fluorochemical phase was collected to afford 2,2,3,3,4,4,4-heptafluorobutyl 1,1,2,2,3,3,4,4,4-nonafluorobutane-1-sulfonate (415 g, 93 mass %, 81% yield). The mass % purity of the desired product was determined by GC-FID analysis. The product was stored over 4 Å molecular sieves and was used for the next step without additional purification.

To a 500 mL 3-necked round-bottom flask equipped with a magnetic stir bar, temperature probe, cold water condenser, and addition funnel was charged diglyme (200 mL). Lithium bromide (70.2 g, 808 mmol) was then added in small portions with a temperature rise to 50° C. observed. Once the temperature had dropped to 35° C., 2,2,3,3,4,4,4-heptafluorobutyl 1,1,2,2,3,3,4,4,4-nonafluorobutane-1-sulfonate (197.1 g, 415 mmol) was added via the addition funnel at a rate which avoided exceeding 40° C. reaction temperature. After complete addition, the resultant reaction mixture was heated to 60° C. with stirring. After 16 hr, the reaction mixture was then cooled to room temperature with stirring followed by the addition of water (200 mL). The fluorous layer was collected and washed again with water (2×100 mL) to give the desired 4-bromo-1,1,1,2,2,3,3-heptafluorobutane (93.7 g, 81 mass %, 71% yield). The isolated material was used without further purification for the next step.

To a two-neck round bottom flask equipped with a water-cooled reflux condenser, magnetic stir bar, and rubber septum were added powdered potassium hydroxide (54.6 g of 85 wt % KOH powder, 827 mmol) and water (70 mL). After addition of water, the temperature had reached >65° C. The solution was allowed to cool to 30° C. with stirring before the addition of tetrabutylammonium chloride (5.0 g, 18 mmol). The resultant mixture was then slowly heated to 35° C. followed by the dropwise addition of 4-bromo-1,1,1,2,2,3,3-heptafluoro-butane (134.3 g at 81 wt % purity, 414 mmol) over the course of 15 minutes via syringe. The resultant amber mixture was then allowed to stir for one hour at the same temperature. GC-FID analysis of the fluorochemical phase indicated approximately 92% conversion of the starting material. After an overnight stir at 35° C., the resultant reaction mixture was allowed to cool to room temperature followed by the addition of 100 mL water. The fluorous phase was separated and analyzed by GC-FID which indicated a mixture containing 88% of the desired 1-bromo-2,3,3,4,4,4-hexafluoro-but-1-ene. Concentric tube distillation (57° C., 740 mm/Hg) afforded the desired 1-bromo-2,3,3,4,4,4-hexafluoro-but-1-ene (85.1 g, 85% yield) as a colorless liquid. The identity of the purified composition was confirmed by GC-MS analysis.

### Example 3: 1-bromo-2,3,3-trifluoroprop-1-ene

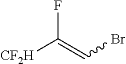

To a 2 L 3-neck round bottom flask equipped with magnetic stirrer, thermocouple, cold water condenser, and an addition funnel were charged 2,2,3,3-tetrafluoropropan-1-ol (200 g, 1.51 mol), PBSF (457.2 g, 1.51 mol), and water (400 mL). There was no observed temperature rise during addition of reagents. The addition funnel was then charged with potassium hydroxide (238 g of a 50% solution in water, 2.12 mol). The 50% potassium hydroxide solution was then added to the stirring mixture dropwise at a rate which kept the internal reaction temperature below 43° C. Once all potassium hydroxide was added, a white hazy mixture was observed. After a 16 hr stir without heating, the resultant reaction mixture was diluted by the addition of water (300 mL). Two layers were observed with white solid at the interface. The bottom layer along with solids were filtered to give a filtrate consisting of mostly fluorochemical layer with some aqueous. The filtrate was then washed with water (300 mL) and the fluorochemical phase was collected to afford 2,2,3,3-tetrafluoropropyl-1,1,2,2,3,3,4,4,4-nonafluorobutane-1-sulfonate (446 g, 87 mass %, 62% yield). The mass % purity of the product was confirmed by GC-FID analysis. The product was stored over 4 Å molecular sieves and was used for the next step without additional purification.

To a 500 mL 3-neck round-bottom flask equipped with a magnetic stir bar, temperature probe, cold water condenser, and addition funnel was charged diglyme (200 mL). Lithium bromide (76.3 g, 879 mmol) was then added in small portions with a temperature rise to 54° C. observed. Once the temperature had dropped to 35° C., 2,2,3,3-tetrafluoropropyl 1,1,2,2,3,3,4,4,4-nonafluorobutane-1-sulfonate (190 g, 86 mass %, 395 mmol) was added via the addition funnel at a rate which avoided exceeding 40° C. reaction temperature. After complete addition, the resultant reaction mixture was heated to 60° C. with stirring. After 16 hr, the reaction mixture was then cooled to room temperature with stirring followed by the addition of water (200 mL). The fluorous layer was collected and washed again with water (2×100 mL) to give the desired 3-bromo-1,1,2,2-tetrafluoropropane (72.4 g, 71 mass %, 69% yield). The isolated material was used without further purification for the next step.

To a two-neck round bottom flask equipped with a water-cooled reflux condenser, magnetic stir bar, and rubber septum were added powdered potassium hydroxide (22.2 g of 85 wt % KOH powder, 336 mmol) and water (55 mL). After addition of water, the temperature had reached >65° C. The solution was allowed to cool to 26° C. with stirring before the addition of tetrabutylammonium chloride (2.1 g, 7.6 mmol). The resultant mixture was then slowly heated to 35° C. followed by the dropwise addition of 3-bromo-1,1,2,2-tetrafluoropropane (54 g at 65 wt % purity, 180 mmol) over the course of 5 minutes via syringe. The resultant amber mixture was then allowed to stir for 16 h at the same temperature. The resultant reaction mixture was then allowed to cool to room temperature followed by the addition of 100 mL water. The fluorochemical phase was separated and analyzed by GC-FID which indicated a mixture containing 54% of the desired 1-bromo-2,3,3-trifluoroprop-1-ene. Concentric tube distillation (75° C., 740 mm/Hg) afforded the desired 1-bromo-2,3,3-trifluoroprop-1-ene (25.1 g, 58% yield) as a colorless liquid. The identity of the purified composition was confirmed by GC-MS analysis.

### Example 4: I-bromo-2,3,3,4,4,5,5-heptafluoropent-1-ene

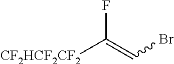

To a 2 L 3-neck round bottom flask equipped with magnetic stirrer, thermocouple, cold water condenser, and an addition funnel were charged 2,2,3,3,4,4,5,5-octafluoropentan-1-ol (200 g, 862 mmol), PBSF (270 g, 894 mmol), and water (400 mL). There was no observed temperature rise during addition of reagents. The addition funnel was then charged with sodium hydroxide (94.2 g of a 50% solution in water, 1.18 mol). The 50% sodium hydroxide solution was then added to the stirring mixture dropwise at a rate which kept the internal reaction temperature below 50° C. Once all sodium hydroxide was added, a white hazy mixture was observed. After a 16 hr stir without heating, the resultant reaction mixture was diluted by the addition of water (300 mL). Two layers were observed with white solid at the interface. The bottom layer along with solids were filtered to give a filtrate consisting of mostly fluorochemical layer with some aqueous. The filtrate was then washed with water (300 mL) and the fluorochemical phase was collected to afford 2,2,3,3,4,4,5,5-octafluoropentyl-1,1,2,2,3,3,4,4,4-nonafluorobutane-1-sulfonate (304.9 g, 85 mass %, 58% yield). The mass % purity of the product was confirmed by GC-FID analysis. The product was stored over 4 Å molecular sieves and was used for the next step without additional purification.

To a 500 mL 3-neck round-bottom flask equipped with a magnetic stir bar, temperature probe, cold water condenser, and addition funnel was charged diglyme (150 mL). Lithium bromide (60.5 g, 697 mmol) was then added in small portions with a temperature rise up to 55° C. observed. Once the temperature had dropped to 35° C., 2,2,3,3,4,4,5,5-octafluoropentyl 1,1,2,2,3,3,4,4,4-nonafluorobutane-1-sulfonate (188 g, 366 mmol) was added via the addition funnel at a rate which avoided exceeding 40° C. reaction temperature. After complete addition, the resultant reaction mixture was heated to 58° C. with stirring. After 16 hr, the reaction mixture was then cooled to room temperature with stirring followed by the addition of water (200 mL). The fluorous layer was collected and washed again with water (2×100 mL) to give the desired 5-bromo-1,1,2,2,3,3,4,4-octafluoropentane (105 g, 80 mass %, 78% yield). The isolated material was used without further purification for the next step.

To a two-neck round bottom flask equipped with a water-cooled reflux condenser, magnetic stir bar, and rubber septum were added powdered potassium hydroxide (45.0 g of 85 wt % KOH powder, 682 mmol) and water (50 mL). After addition of water, the temperature had reached >65° C. The solution was allowed to cool to 30° C. with stirring before the addition of tetrabutylammonium chloride (4.5 g, 16 mmol). The resultant mixture was then slowly heated to 35° C. followed by the dropwise addition of 5-bromo-1,1,2,2,3,3,4,4-octafluoropentane (100 g at 80 wt % purity, 271 mmol) over the course of 5 minutes via syringe. The resultant amber mixture was then heated to 60° C. and was allowed to stir for 16 h at the same temperature. The resultant reaction mixture was then allowed to cool to room temperature followed by the addition of 100 mL water. The fluorochemical phase was separated and analyzed by GC-FID which indicated >99% conversion of the 5-bromo-1,1,2,2,3,3,4,4-octafluoropentane starting material and a mixture containing 95% of the desired 1-bromo-2,3,3,4,4,5,5-heptafluoropent-1-ene. Concentric tube distillation (110° C., 740 mm/Hg) afforded the desired 1-bromo-2,3,3,4,4,5,5-heptafluoropent-1-ene (41.4 g, 70% yield) as a colorless liquid. The identity of the purified composition was confirmed by GC-MS analysis.

### Example 5: 1-chloro-2,3,3,4,4,5,5,5-pent-1-ene

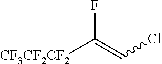

To a 5-L 3-neck round bottom flask equipped with mechanical stirrer, thermocouple, cold water condenser and an addition funnel were charged 2,2,3,3,4,4,5,5,5-nonafluoropentan-1-ol (500 g, 2.0 mol), PBSF (604 g 2.0 mol) and 2500 g water. 337 g of 50% KOH was slowly added via the addition funnel at a rate to keep the temperature below 35° C. The reaction mixture was stirred for 16 hr at room temperature. The reaction mixture was filtered, and the filtrate was added to a separatory funnel. The lower phase still contained 13.3% unreacted 2,2,3,3,4,4,5,5,5-nonafluoropentan-1-ol and 4.0% PBSF. The reaction mixture was charged to a 2-L round bottom flask equipped as above and 500 mL water, 110 g PBSF, and 81 g of 50% KOH were added. The mixture was stirred for 2 hours, filtered, phase separated and water washed to afford 625 g of 2,2,3,3,4,4,5,5,5-nonafluoropentyl 1,1,2,2,3,3,4,4,5-nonafluoropentane-1-sulfonate with a purity by gas chromatography of 99%.

To a 1-L round bottom flask equipped with magnetic stirrer, water condenser, N2 bubbler, thermocouple and addition funnel were charged 1600 g dimethylformamide and lithium chloride (25.7 g, 0.61 mol). An exotherm was observed. The flask was cooled to room temperature and 2,2,3,3,4,4,5,5,5-nonafluoropentyl 1,1,2,2,3,3,4,4,5-nonafluoropentane-1-sulfonate (215 g, 0.40 mol) was added and the flask was stirred at room temperature for 48 hours. One L of water was added and the mixture was steam distilled to afford 105.2 g of 5-chloro-1,1,1,2,2,3,3,4,4-nonafluoro-pentane with a purity by gas chromatography of 98.8%. GC-MS confirmed the structure.

To a 200 mL round bottom flask equipped with magnetic stirrer, water condenser, N2 bubbler, thermocouple and heating mantle were charged 23.2 g of 90% KOH and 25 g water. The flask was cooled to room temperature. 2.5 g tetrabutylammonium chloride and 5-chloro-1,1,1,2,2,3,3,4,4-nonafluoro-pentane (50 g, 0.19 mol) were added and the temperature was kept below 35° C. with an ice bath around the flask. The mixture was stirred for 1 hour and phase separated to afford 41 g of 1-chloro-2,3,3,4,4,5,5,5-pent-1-ene with a purity of 96.8%. The 1-chloro-2,3,3,4,4,5,5,5-pent-1-ene was combined with other batches prepared as above and fractionated to a purity of 99.9% as determined by F-NMR. Boiling point was about 64° C.

### Example 6: 1-chloro-2, 3,3,4,4,4-but-1-ene

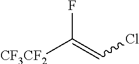

To a 3-L 3-neck round bottom flask equipped with mechanical stirrer, thermocouple, cold water condenser and an addition funnel were charged 2,2,3,3,4,4,4-heptafluorobutan-1-ol (350 g, 1.75 mol), PBSF (528 g, 1.75 mol) and 700 g water. 300 g of 50% KOH was slowly added via the addition funnel at a rate to keep the temperature below 35° C. The reaction mixture was stirred for 16 hr at room temperature. The reaction mixture was filtered and the filtrate added to a separatory funnel. The lower phase was water washed to afford 2,2,3,3,4,4,4-heptafluorobutyl 1,1,2,2,3,3,4,4,4-nonafluorobutane-1-sulfonate (502 g 69% purity by GC.). The material was re-treated with 200 ml water and 50 g of 50% KOH and stirred for two hours. Phase separated and water wash afforded 461 g 2,2,3,3,4,4,4-heptafluorobutyl 1,1,2,2,3,3,4,4,4-nonafluorobutane-1-sulfonate. Purity by gas chromatography was 95.5%.

To a 3-L round bottom flask equipped with overhead stirrer, water condenser, N2 bubbler, thermocouple and addition funnel were charged 1800 mL dimethylformamde and lithium chloride (126.6 g, 2.98 mol). An exotherm was observed. The flask was cooled to room temperature and 2,2,3,3,4,4,4-heptafluorobutyl 1,1,2,2,3,3,4,4,4-nonafluorobutane-1-sulfonate (430 g, 0.89 mol) was added and the flask was heated to 50° C. and held 16 hours. One L of water was added and the mixture was steam distilled to afford 174.6 g of 4-chloro-1,1,1,2,2,3,3-heptafluoro-butane with a purity by gas chromatograph of 99.0%. GC-MS confirmed the structure.

To 1-L round bottom flask equipped with magnetic stirrer, water condenser, N2 bubbler, thermocouple and addition funnel was charged sodium methoxide (50.4 g, 0.93 mol) dissolved in methanol (198 g, 6.2 mol). 4-chloro-1,1,1,2,2,3,3-heptafluoro-butane (170 g, 0.78 mol) was added and the mixture heated to 50° C. After 24 hours, 38% of the starting material remained. An additional identical charge of sodium methoxide in methanol was added and held an additional 2 hr at 50° C. The flask was cooled to room temperature and water was added. The lower phase was separated and fractionated to afford 27.4 g at >98.5% purity of 1-chloro-2,3,3,4,4,4-hexafluoro-but-1-ene with a boiling point of about 37° C. The structure was confirmed by GC/MS and F-NMR.

### Example 7: 1-chloro-2,3,3-trifluoro-3-(trifluoromethoxy)prop-1-ene

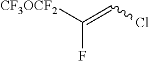

To a 500 mL round bottom flask equipped with a dry ice bath, nitrogen bubbler, PFTE tubing with SWAGELOK fittings, condenser connected to a chiller, magnetic stir bar, and thermocouple, were charged sodium borohydride (37 g, 977.993 mmol) and diethylene glycol dimethyl ether (187.4 g, 1397 mmol). The flask was then chilled to −40° C., the condenser was set to −15° C., and perfluoromethoxypropionyl fluoride (214.9 g, 907.7 mmol), was fed, using the PTFE tube with SWAGELOK fittings, slowly from an inverted cylinder over 4 hours. The reaction was kept below −15° C. during the addition. When the addition was complete the dry ice bath was removed, and the reaction was stirred 16 hours. The reaction was quenched with methanol (98 g, 3058.52 mmol) over 2 hours. The reactor was stirred an additional 30 minutes until off-gassing ceased. The reaction material was then transferred to a 1000 mL round bottom flask equipped with overhead stirrer. While stirring the reaction, 200 mL of water were added to the flask followed by phosphoric acid (260 g, 928.631 mmol, 35 mass %) from an addition funnel over the course of 45 min. The reaction was heated to 50° C., stirred for a half hour, then cooled on dry ice, phase separated and the lower phase washed with water 3 times. 216 g of material at 48 GC-FID area % 2,2,3,3-tetrafluoro-3-(trifluoromethoxy) propan-1-ol was recovered. The recovered material was washed another 4 times. 124 g of 2,2,3,3-tetrafluoro-3-(trifluoromethoxy) propan-1-ol (124 g, 573.94 mmol, 63.23% yield) at 76 GC-FID area % was recovered.

To a 500 mL round bottom flask equipped with Claisen adapter, water condenser, addition funnel thermocouple and overhead stirrer were charged 2,2,3,3-tetrafluoro-3-(trifluoromethoxy) propan-1-ol (50 g, 175.89 mmol, 76 mass %), nonafluorobutanesulfonyl (1.05 equivalent, 184.68 mmol), water (70.2 g, 3900 mmol). An approximate 50 wt % KOH solution was prepared by dissolving potassium hydroxide (29 g, 516.889 mmol) in 30.2 g of water. The resulting solution was added dropwise via the addition funnel. During the addition a dry ice and water bath was used to keep the reaction temperature below 35° C. After the addition the reaction was stirred 16 hr at room temperature. A sample of the reaction was water washed and GC analysis revealed 12 GC-FID area % of unconverted alcohol. An additional 15.8 g PBSF were added and the reaction was stirred for 2 hours. A final GC analysis showed 2 area % unconverted alcohol by GC-FID. [2,2,3,3-tetrafluoro-3-(trifluoromethoxy) propyl] 1,1,2,2,3,3,4,4,4-nonafluorobutane-1-sulfonate (42 g, 69.137 mmol, 39.308% yield) was recovered with a purity by GC of 82% after filtration with diatomaceous earth to remove solids. The recovered material was dried over molecular sieves.

To a 250 mL round bottom flask equipped with magnetic stir plate, water condenser, thermocouple, and addition funnel were charged lithium chloride (9.62 g, 227 mmol) and N,N,-dimethylformamide (24.1 g, 330 mmol) which were stirred until a suspension was made. An additional 20 mL of dimethyl formamide (DMF) was added to help break up the lithium chloride. [2,2,3,3-tetrafluoro-3-(trifluoromethoxy) propyl]1,1,2,2,3,3,4,4,4-nonafluorobutane-1-sulfonate (34.5 g, 56.8 mmol, 82%) was charged via an addition funnel. The addition proceeded slowly at first, but the rate of addition was increased as no exothermic reaction occurred. The reaction was left to stir and after 4 hr a small sample was water washed, filtered, and analyzed by GC. The reaction had converted all but 7 GC-FID area % of the nonaflate so the reaction was stirred an additional 16 hours. The reaction was quenched with water, transferred to a separatory funnel and the lower phase collected. A total of 10 g of 3-chloro-1,1,2,2-tetrafluoro-1-(trifluoromethoxy) propane (10 g, 35.394 mmol) was recovered at 83 GC-FID area %.

To a 50 mL round bottom flask equipped with thermocouple, magnetic stirrer, water condenser, and addition funnel, was charged 3-chloro-1,1,2,2-tetrafluoro-1-(trifluoromethoxy) propane (10.5 g, 44.8 mmol, 100 mass %), and tetrabutylammonium chloride (0.5 g, 2 mmol, 100 mass %). Using an addition funnel, potassium hydroxide (12.11 g, 107.9 mmol, 50 mass %) was charged to the reaction slowly over 20 minutes during which time the reaction turned a yellow/orange color. After the addition was complete a slight exotherm was seen (33.3° C.). At 40 minutes a sample was taken and analyzed by GC. The results showed partial conversion to the olefin. The reaction was stirred 16 hr after which the reaction was quenched with water, phase separated, and water washed. The material was passed through a 0.2 μm syringe filter and analyzed by GC. The GC showed complete conversion of the starting material and the desired material at 83 GC-FID area %. The material was then purified by distillation and analyzed by GC which resulted in 99.6 GC-FID area % of a possible desired material. GC-MS data of the 4.6 g of crude material confirmed the formation of 1-chloro-2,3,3-trifluoro-3-(trifluoromethoxy) prop-1-ene (2.6 g, 12 mmol).

### Example 8: 4-(2-chloro-1-fluoro-vinyl)-2,2,3,3,5,5,6,6-octafluoro-morpholine

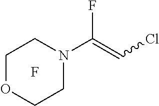

In a 3 L 3-neck round-bottom flask equipped with overhead stirring, thermocouple, cold water condenser, dry N2 line and an addition funnel, tetraethylene glycol dimethyl ether (800 g, 3.60 mol) and sodium borohydride (87.0 g, 2.30 mol) were charged. The mixture was stirred for 30 min to dissolve most of sodium borohydride. A dry ice-water bath was added to cool down the reaction mixture. When the reaction temperature dropped to 10° C., 2,2-difluoro-2-(2,2,3,3,5,5,6,6-octafluoromorpholin-4-yl)acetyl fluoride (699.4 g, 2.14 mol), prepared by electrochemical fluorination of 4-morpholineethanol via a Simons ECF cell of essentially the type described U.S. Pat. No. 2,713,593 and in R. E. Banks, Preparation, Properties and Industrial Applications of Organofluorine Compounds, pages 19-43, Halsted Press, New York (1982), was added via the addition funnel at such a rate to keep reaction temperature below 65° C. Once the addition of 2,2-difluoro-2-(2,2,3,3,5,5,6,6-octafluoromorpholin-4-yl)acetyl fluoride was complete, the reaction mixture was heated to 80° C. and stirred overnight. The reaction mixture was cooled to room temperature and quenched by slow addition of methanol (155.8 g, 4.86 mol). The reaction mixture was then heated to 50° C. until off gassing stopped. Then 1000 mL 35% H₃PO₄ was added. The reaction mixture was heated to 50° C. to dissolve formed salts. The reaction mixture was separated in a separatory funnel and the lower fluorochemical phase water washed. 697 g crude product was obtained. GC-MS data show that the crude product contained 74% of the desired 2,2-difluoro-2-(2,2,3,3,5,5,6,6-octafluoromorpholin-4-yl)ethanol. The resulting crude product was distilled from polyphosphoric acid.

In a 3 L 3-neck round-flask equipped with magnetic stirrer, thermocouple, cold water condenser, and addition funnel, 2,2-difluoro-2-(2,2,3,3,5,5,6,6-octafluoromorpholin-4-yl)ethanol (434.0 g, 1.40 mol), 1,1,2,2,3,3,4,4,4-nonafluorobutane-1-sulfonyl fluoride (442.3 g, 1.46 mol) and water (560 g, 31.09 mol) were charged, no obvious exotherm was observed. 50% KOH (300.3 g, 2.68 mol) aqueous solution was then added via the addition funnel at such a rate to keep the internal reaction temperature below 35° C. Once all KOH was added, the reaction mixture was stirred at 35° C. for three days. The reaction mixture was transferred to a separatory funnel, the resulting fluorochemical phase was separated and was washed with water twice, and 779 g crude product was obtained. GC-MS data show 96% of desired [2,2-difluoro-2-(2,2,3,3,5,5,6,6-octafluoromorpholin-4-yl)ethyl]1,1,2,2,3,3,4,4,4-nonafluorobutane-1-sulfonate.

In a 2000 mL 3-neck round-bottom flask equipped with magnetic stirring, thermocouple, cold water bath, cold water condenser, dry N2 line and addition funnel, lithium chloride (55.6 g, 1.31 mol) and DMF (600 g, 8.21 mol) were mixed. Once the exotherm had subsided, [2,2-difluoro-2-(2,2,3,3,5,5,6,6-octafluoromorpholin-4-yl)ethyl]1,1,2,2,3,3,4,4,4-nonafluorobutane-1-sulfonate (738 g, 1.24 mol) was added via the addition funnel while keeping the reaction temperature below 40° C. Once the addition was complete, the mixture was stirred at 60° C. overnight. The reaction mixture was cooled to room temp and distilled. 406 g crude product was obtained. GC/MS data show that it contained 97% of the desired 4-(2-chloro-1,1-difluoro-ethyl)-2,2,3,3,5,5,6,6-octafluoro-morpholine.

To a 1000 mL 3-neck round-bottom flask equipped with magnetic stirrer, thermocouple, cold water condenser and addition funnel, KOH (85%, 314 g, 4.76 mol) and water (318 g, 17.65 mol) were added. Once the exotherm subsided, tetrabutylammonium chloride (7.4 g, 0.03 mol) was added. Then 4-(2-chloro-1,1-difluoro-ethyl)-2,2,3,3,5,5,6,6-octafluoro-morpholine (278 g, 0.84 mol) was added via the addition funnel while keeping the reaction temperature below 20° C. Once the addition of 4-(2-chloro-1,1-difluoro-ethyl)-2,2,3,3,5,5,6,6-octafluoro-morpholine was complete, the reaction mixture was heated to 60° C. for two days. The crude product was steam distilled and 74 g product was obtained, GC-MS data show it contained 93% of the desired 4-[(E/Z)-2-chloro-1-fluoro-vinyl]-2,2,3,3,5,5,6,6-octafluoro-morpholine.

### Example 9: 3-chloro-1,1,2-trifluoro-N-(1,1,2,2,2-pentafluoroethyl)-N-(trifluoromethyl)prop-2-en-1-amine

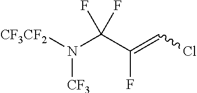

In a 3 L 3-neck round-bottom flask equipped with overhead stirring, thermocouple, cold water condenser, dry N2 line and addition funnel, tetraethylene glycol dimethyl ether (201 g, 0.90 mol) and sodium borohydride (33 g, 0.87 mol) were charged. An exotherm was observed. The mixture was stirred for 30 minutes to dissolve most of the sodium borohydride. A dry ice-water bath was added to cool the reaction mixture. When reaction temperature dropped to 10° C., 2,2,3,3-tetrafluoro-3-[1,1,2,2,2-pentafluoroethyl(trifluoromethyl)amino]propanoyl fluoride (290 g, 0.83 mol), prepared by electrochemical fluorination of methyl 3-[ethyl(methyl)amino]propanoate via a Simons ECF cell of essentially the type described U.S. Pat. No. 2,713,593 and in R. E. Banks, Preparation, Properties and Industrial Applications of Organofluorine Compounds, pages 19-43, Halsted Press, New York (1982), was added via the addition funnel at such a rate to keep reaction temperature below 65° C. Once the addition of 2,2,3,3-tetrafluoro-3-[1,1,2,2,2-pentafluoroethyl(trifluoromethyl)amino]propanoyl fluoride was complete, the reaction mixture was heated to 80° C. and stirred overnight. The reaction mixture was quenched with a slow addition of methanol (61.96 g, 1.93 mol). The reaction mixture was heated to 50° C. until no more off gassing was seen. 450 ml 35% H₃PO₄ was added the reaction mixture was heated to 50° C. to dissolve salts that were formed. The reaction mixture was transferred to a separatory funnel, and the lower phase was separated and water washed. 183 g crude product, verified by GC-MS was obtained.

In a 1000 mL 3-neck round bottom flask equipped with magnetic stirring, thermocouple, cold water condenser and addition funnel, the 2,2,3,3-tetrafluoro-3-[1,1,2,2,2-pentafluoroethyl(trifluoromethyl)amino]propan-1-ol (150 g, 0.45 mol), 1,1,2,2,3,3,4,4,4-nonafluorobutane-1-sulfonyl fluoride (138 g, 0.46 mmol), water (300 g, 16.65 mol) were combined. The KOH (55.5 g, 0.50 mol, 50 wt %) was added dropwise via the addition funnel at such a rate to maintain the temperature in the flask at or below 35° C. After stirring for 16 hr the reaction mixture was filtered and transferred to a separatory funnel. Gas chromatography showed about a 50% conversion. The fluorochemical phase was transferred to a 600 mL Parr reactor with an additional KOH charge and stirred 16 hr at room temperature to provide complete conversion. The reaction mixture was phase separated and filtered. 161 g crude product was obtained. GC-MS data of crude product show it contained 89% of the desired [2,2,3,3-tetrafluoro-3-[1,1,2,2,2-pentafluoroethyl(trifluoromethyl)amino]propyl] 1,1,2,2,3,3,4,4,4-nonafluorobutane-1-sulfonate.

To a 2 L 3-neck round bottom flask equipped with magnetic stirrer, thermocouple, cold water condenser and addition funnel, lithium chloride (66 g, 1.56 mol) and DMF (825 ml) were mixed. [2,2,3,3-tetrafluoro-3-[1,1,2,2,2-pentafluoroethyl(trifluoromethyl)amino]propyl] 1,1,2,2,3,3,4,4,4-nonafluorobutane-1-sulfonate (161 g, 0.26 mol) was added and the mixture was stirred 57° C. for two days. Crude product was distilled by a Dean Stark Trap. 40 g crude product was obtained. GC data show that the crude product contained 80% of the desired 3-chloro-1,1,2,2-tetrafluoro-N-(1,1,2,2,2-pentafluoroethyl)-N-(trifluoromethyl)propan-1-amine.

To a 100 mL 3-neck round-bottom flask equipped with magnetic stirrer, thermocouple, cold water condenser and addition funnel, KOH (85%, 12.7 g, 0.19 mol) and water (12.7 g, 0.71 mol) were mixed. An exotherm was observed. Once the exotherm subsided, tetrabutylammonium chloride (0.9 g, 0.004 mol) was added. 3-chloro-1,1,2,2-tetrafluoro-N-(1,1,2,2,2-pentafluoroethyl)-N-(trifluoromethyl)propan-1-amine (33.7 g, 0.096 mol) was then slowly added into reaction flask via the addition funnel, keeping the reaction temperature below 20° C. Once the addition of 3-chloro-1,1,2,2-tetrafluoro-N-(1,1,2,2,2-pentafluoroethyl)-N-(trifluoromethyl)propan-1-amine was complete. The reaction mixture was heated to 60° C. and held 16 hr. 19 g of crude product was obtained by steam distillation. GC-MS data show that the crude product contained 92% of the desired (E/Z)-3-chloro-1,1,2-trifluoro-N-(1,1,2,2,2-pentafluoroethyl)-N-(trifluoromethyl)prop-2-en-1-amine.

### Example 10: 1-chloro-2,3,4,4,4-pentafluoro-but-1-ene

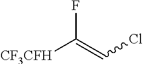

To a 5-L 3-neck round bottom flask equipped with mechanical stirrer, thermocouple, cold water condenser and an addition funnel were charged 2,2,3,4,4,4-hexafluorobutan-1-ol (469 g, 2.58 mol), PBSF (783 g, 2.59 mol) and 2500 g water. 475 g of 50% KOH was slowly added via the addition funnel at a rate to keep the temperature below 35° C. The reaction mixture was stirred for 16 hr at room temperature. The reaction mixture was filtered and the filtrate was added to a separatory funnel. The lower phase was water washed and phase separated to afford 911 g of 2,2,3,4,4,4-hexafluorobutyl 1,1,2,2,3,3,4,4,4-nonafluorobutane-1-sulfonate with a GC purity of 93.5%. Low boiling materials were then removed by rotary evaporation at 50° C. and 20 torr, resulting in a purity of 97.0%.

To a 5-L round bottom flask equipped with overhead stirrer, water condenser, N2 bubbler, thermocouple and addition funnel were charged 2325 g dimethylformamide and lithium chloride (186.3 g, 4.4 mol). An exotherm was observed. The flask was cooled to room temperature and 2,2,3,4,4,4-hexafluorobutyl 1,1,2,2,3,3,4,4,4-nonafluorobutane-1-sulfonate (816 g, 1.76 mol) was added. The flask was then heated to 50° C. and held at that temperature for 16 hr. Water was added to the flask and the contents were steam distilled. The lower fluorochemical phase was phase separated and water washed. The batch was repeated and the fluorochemical phases combined to afford 611 g of 4-chloro-1,1,1,2,3,3-hexafluoro-butane. Structure was confirmed by GC-MS.

To a 1-L round-bottom flask equipped with magnetic stirrer, water condenser, N2 bubbler, thermocouple and heating mantle were charged 323 g of 25 wt. % sodium methoxide in methanol. 4-chloro-1,1,1,2,3,3-hexafluoro-butane (200 g, 1.0 mol) were added and the temperature rose to 64° C. The flask was cooled to 50° C. and held for 1 hr, then cooled to room temperature and held for 16 hr. Gas chromatography showed about 20% unconverted starting material. An additional 0.5 equivalents of 25 wt. % sodium methoxide in methanol was added and the mixture heated to 50° C. About 4% unconverted starting material remained. 250 mL of water was added to the flask and the contents steam distilled to provide 134 g of 1-chloro-2,3,4,4,4-pentafluoro-but-1-ene with a purity by gas chromatography of 53.6%. The material was fractionated to afford 26.1 g with a purity of >95.0%. Structure and purity were determined by GC-MS and F-NMR. The boiling point was approximately 63° C.

### Example 11: 1-chloro-2,3-difluoro-3-(trifluoromethoxy)prop-1-ene

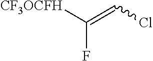

To a 600 mL Parr reactor were charged methanol (162 g, 5055.93 mmol) and tert-butyl peroxy-2-ethylhexanoate (6.7 g, 31 mmol). The reactor was then sealed and heated to 70° C. Trifluoromethyl trifluorovinylether (190 g, 1.14 mol) was slowly added from a cylinder. After 124 g of trifluoromethyl trifluorovinylether was added, the addition was stopped and the reactor held at 70° C. for 16 hr. The reactor was cooled in a dry ice-acetone bath and another 7.5 g of the initiator was added. The reactor was then heated to 75° C. and an additional 66 g of trifluoromethyl trifluorovinylether were charged. The reactor was held at 75 C° for 16 hr, cooled to room temperature and residual pressure vented. The reactor contents were water washed and the lower phase separated to afford 230 g of material. GC-MS confirmed main peak is 2,2,3-trifluoro-3-(trifluoromethoxy)propan-1-ol.

To a 1000 mL round bottom flask equipped with a water condenser, magnetic stirrer, dry ice bath, and addition funnel were charged 2,2,3-trifluoro-3-(trifluoromethoxy)propan-1-ol (228 g, 1151.2 mmol), nonafluorobutanesulfonyl fluoride (384 g, 1271.15 mmol) and water (233 g, 12933.8 mmol). The flask was then placed in a dry ice bath and 260 g of 50% potassium hydroxide was added drop wise to the reaction flask using an addition funnel. The rate was adjusted to keep the reaction temp below 35° C. After the addition was finished, the dry ice bath was removed and a heating mantle was added. The reaction was stirred 16 hr at 30° C. The flask was then cooled and the contents vacuum filtered into a 1000 mL round bottom flask on dry ice. The recovered material was then water washed and 337 g of material were recovered. GC analysis showed 90% [2,2,3-tetrafluoro-3-(trifluoromethoxy) propyl] 1,1,2,2,3,3,4,4,4-nonafluorobutane-1-sulfonate.

To a 1000-mL round bottom flask equipped with Claisen adapter, thermocouple, magnetic stir plate, water condenser and addition funnel were charged lithium chloride (77.7 g, 1830 mmol) and N,N-dimethylformamide (256.3 g, 3506 mmol). The reaction flask was cooled to room temperature. After the initial exotherm, [2,2,3-tetrafluoro-3-(trifluoromethoxy) propyl] 1,1,2,2,3,3,4,4,4-nonafluorobutane-1-sulfonate (298 g, 620.83 mmol) was added to the addition funnel and added dropwise to the reaction flask keeping the temperature under 45° C. The reaction was cooled to room temperature and stirred for 16 hr. The reaction was quenched with water and GC analysis of the lower phase showed 75% of 3-chloro-1,2,2-trifluoro-1-(trifluoromethoxy)propane. The material was steam distilled to afford 120 g of 3-chloro-1,2,2-trifluoro-1-(trifluoromethoxy)propane with a purity of 94.0%.

To a 250 mL round bottom flask were charged water (30 g, 1665.30 mmol) and potassium hydroxide (30 g, 534.713 mmol). After the KOH had dissolved and the flask cooled to 60° C., tetrabutylammonium chloride (0.8 g, 3 mmol, 100 mass %) was added. 3-chloro-1,2,2-trifluoro-1-(trifluoromethoxy)propane (53 g, 244.79 mmol) was added while the pot temperature was held at 50° C. The heat was removed from the reaction after the addition and the flask cooled to room temperature. The reaction was quenched with water and the product was recovered by steam distillation to afford 45 g 3-chloro-1,2-trifluoro-1-(trifluoromethoxy)prop-3-ene, 94.6% purity. The product is a mixture of E and Z isomers. Structure and purity were determined by GC-MS and F-NMR.

### Results

Table 2 summarizes results of Largest Soluble Hydrocarbon (LSH) testing of Examples 1-11. Since the largest hydrocarbon used was C-23 (C₂₃H₄₈), an LSH of “>23” indicates that the hydrofluoroolefin was miscible with C₂₃H₄₈ without exhibiting haze. The results presented in Table 2 indicate that the hydrofluoroolefins of the present invention are highly suitable fluids for cleaning applications.

TABLE 2
Largest Soluble Hydrogen

| | LSH (n in CₙH₂ₙ₊₂) | |
|---|---|---|
| Example | 25° C. | 50° C. |
| 1 | 19 | >23 |
| 2 | 19 | >23 |
| 3 | 19 | >23 |
| 4 | 19 | >23 |
| 5 | 17 | 22 |
| 6 | 19 | |
| 7 | 19 | >23 |
| 8 | 14 | 18 |
| 9 | 13 | 17 |
| 10 | 19 | >23 |
| 11 | 18 | >23 |

The atmospheric lifetimes of Examples 1-3 were determined from their rates of reaction with hydroxyl radicals as described above and are reported in Table 3.

TABLE 3
Atmospheric Lifetime

| Example | Atmospheric Lifetime |
|---|---|
| 1 | Degrades by direct photolysis and |
| | ozonolysis. Likely <0.05 years |
| 2 | Degrades by direct photolysis and |
| | ozonolysis. Likely <0.05 years |
| 3 | Degrades by direct photolysis and |
| | ozonolysis. Likely <0.05 years |

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

1. A hydrofluoroolefin compound represented by the following general formula (II):
R_{f}(CFH)ₙCF═CHX (II)
where R_{f} is a linear, branched, or cyclic perfluoroalkyl group having 1-6 carbon atoms, and optionally comprises at least one catenated heteroatom selected from nitrogen or oxygen;
n is 0 or 1;
X is Cl or Br;
with the following proviso:
when R_{f} is CF3, then n is 1.

2. The hydrofluoroolefin compound of claim 1, wherein the hydrofluoroolefin compound has the following general formula (IIA):
RfCF═CHCl (IIA)
where R_{f} is a linear, branched, or cyclic perfluoroalkyl group having 2-6 carbon atoms, and optionally comprises at least one catenated heteroatom selected from nitrogen or oxygen.

3. The hydrofluoroolefin compound of claim 1, wherein the hydrofluoroolefin compound has the following general formula (IIB):
RfCF═CHCl (IIB)
where R_{f} is a perfluoroalkyl group having 2-3 carbon atoms.

4. The hydrofluoroolefin compound of claim 1, wherein the hydrofluoroolefin compound has the following general formula (IIC):
RfCF═CHBr (IIC)
where R_{f} is a linear, branched, or cyclic perfluoroalkyl group having 2-6 carbon atoms, and optionally comprises at least one catenated heteroatom selected from nitrogen or oxygen.

5. The hydrofluoroolefin compound of claim 1, wherein the hydrofluoroolefin compound has the following general formula (IID):
RfCF═CHBr (IID)
where R_{f} is a perfluoroalkyl group having 2-3 carbon atoms.

6. The hydrofluoroolefin compound of claim 1, wherein the hydrofluoroolefin compound has a solubility factor greater than 0.

7. A composition comprising:
a hydrofluoroolefin represented by the following structural formula (I):
following structural formula (I):
(H)ₙ—R_{f}—(CFH)ₘ—CF═CHX (I)
where R_{f} is a linear, branched, or cyclic perfluoroalkyl group having 1-6 carbon atoms, and optionally comprises at least one catenated heteroatom selected from nitrogen or oxygen;
n is 0 or 1;
m is 0 or 1;
m+n=0 or 1; and
X is Cl or Br;
with the following provisos:
when X is Cl and R_{f} is CF3, then m is 1;
when X is Br and R_{f} is CF₃, then m is 1; and
when R_{f} is cyclic, then m+n=0; and
a contaminant;
wherein the hydrofluoolefin is present in the composition at an amount of at least 25% by weight, based on the total weight of the composition.

8. The composition of claim 7, wherein the contaminant comprises a long chain hydrocarbon alkane.

9. The composition of claim 7, wherein the hydrofluoroolefin compound has the following general formula (IA):
CF₂HCF₂CF₂CF═CHCl; (IA).

10. The composition of claim 7, wherein the hydrofluoroolefin compound has the following general formula (IB):
CF₂H(CF₂)ₙCF═CHBr (IB)
where n is 0 or 2.

11. The composition of claim 7, wherein the hydrofluoroolefin compound has a solubility factor greater than 0.

12. A process for removing a contaminant from a substrate, the process comprising the steps of:
contacting the substrate with a hydrofluoroolefin represented by the following structural formula (I):
(H)ₙ—R_{f}—(CFH)ₘ—CF═CHX (I)
where R_{f} is a linear, branched, or cyclic perfluoroalkyl group having 1-6 carbon atoms, and optionally comprises at least one catenated heteroatom selected from nitrogen or oxygen;
n is 0 or 1;
m is 0 or 1;
m+n=0 or 1; and
X is Cl or Br;
with the following provisos:
when X is Cl and R_{f} is CF3, then m is 1;
when X is Br and R_{f} is CF₃, then m is 1; and
when R_{f} is cyclic, then m+n=0;
wherein the contaminant comprises a long chain hydrocarbon alkane.
